(19) United States
(12) Patent Application Publication
(10) Pub. No.: US 2010/0184016 A1
(43) Pub. Date: Jul. 22, 2010

(54) METHODS AND COMPOSITIONS IN THE TREATMENT OF PORCINE CIRCOVIRAL INFECTION

(76) Inventors: David Jacques Gerard Lefebvre, Gentbrugge (BE); Hans Nauwynck, Zomergem (BE)

Correspondence Address: CASTELLANO PLLC, P.O. Box 1555, Great Falls, VA, 22066, US

(21) Appl. No.: 12/665,938

(22) PCT Filed: Jun. 19, 2008
(86) PCT No.: PCT/EP08/04942

§ 371 Date: Feb. 10, 2010

(30) Foreign Application Priority Data
Jun. 22, 2007 (GB) 07/12160.1

(51) Int. Cl. C12Q 1/70; C07K 16/08; C12N 7/02

(52) U.S. Cl. 435/5; 530/387.9; 435/239

(57) ABSTRACT

The invention relates generally to the field of virology. More particularly, the present invention relates to methods of diagnosing, prognosis, treatment and prevention of porcine circoviral infection in mammals, in particular of porcine circovirus type 2 (PCV2).

Methods of using a nucleic acid(s) and/or a protein(s), which are immunogenic in said mammal, and antibodies immunospecific for said protein(s), to treat, diagnose and/or prevent said porcine circoviral infection, are provided for by the present invention.

### FIELD OF THE INVENTION

The invention relates generally to the field of virology. More particularly, the present invention relates to methods of diagnosing, prognosis, treatment and prevention of porcine circoviral infection in mammals, in particular of porcine circovirus type 2 (PCV2).

Methods of using a nucleic acid(s) and/or a protein(s), which are immunogenic in said mammal, and antibodies immunospecific for said protein(s), to treat, diagnose and/or prevent said porcine circoviral infection, are provided for by the present invention.

### BACKGROUND TO THE INVENTION

Porcine circovirus type 2 (PCV2) is widespread in domestic and wild pigs. It belongs to the family of the Circoviridae. Another member of that family, porcine circovirus type 1 (PCV1) was discovered and characterized as a non-cytopathic contaminant of the continuous porcine kidney cell line PK-15 ATCC-CCL33.

PCV1 is not regarded as a pathogen for pigs (Tischer et al., 1986), whereas PCV2 is considered as the crucial pathogen in postweaning multisystemic wasting syndrome (PMWS), a multifactorial swine disease that causes wasting and death in weaned piglets (Allan and Ellis, 2000). Besides wasting, PCV2 may also cause reproductive failure (West et al., 1999). PCV2 has also been isolated from pigs with porcine dermatitis and nephropathy syndrome (PDNS) and a various number of other diseases, but neither PDNS nor these other diseases have been reproduced experimentally.

The PCV2 virion measures approximately 17 nm in diameter, is non-enveloped and consists of a circular single-stranded DNA surrounded by an icosahedral capsid (Allan et al., 1998). The ambisense DNA molecule contains about 1.77 kilobases and 11 putative open reading frames (ORFS).

Proteins encoded by 3 of these ORFs are considered to play a role in the pathogenesis of PCV2 infections.

ORF1 encodes for the replication associated proteins Rep and Rep'. ORF2 encodes for the 27.8 kDa capsid protein (Hamel et al., 1998). The ORF2 protein is the only structural protein. The ORF3 protein has a molecular mass of 11.8 kDa and has recently been associated with apoptosis in vitro and with PMWS-like lesions in mice (Liu et al., 2006).

Meerts et al. (2005a) demonstrated biological differences between different PCV2 strains in vitro. Replication kinetics of PMWS- and PDNS-associated PCV2 strains were significantly different from reproductive failure-associated PCV2 strains.

Recently, it was demonstrated that the virulence of a PCV2 isolate originating from a PMWS-affected animal differed significantly from an isolate recovered from a subclinically infected animal. Important differences in serologic profile, virus replication and severity of lesions were shown after experimental inoculation of specific pathogen-free pigs (Opriessnig et al., 2006). Several studies mentioned that genetic differences in PCV2 are associated with the geographic region from which the isolates originated and a recently proposed classification system (Olvera et al., 2007) divides PCV2 into two genotypes (1 and 2) and eight clusters (1A to 1C and 2A to 2E). Although several antigenic domains have been discovered on the capsid protein (Mahé et al., 2000), no association has been established so far between the sequence of the capsid and the pathogenicity and antigenicity of a PCV2 strain. Until now, mouse monoclonal antibodies (mAbs) directed against PCV2 did not show major differences in reactivity to different PCV2 strains (McNeilly et al., 2001).

In this study, mAbs to PCV2 were produced, characterized and used to identify serotypes between PCV2 strains, and allows amongst others to differentiate PCV2 strains originating from different clinical presentations and different geographic regions. Using mAbs and the antigenic differences thus identified it now becomes possible to develop a rapid, cheap, consumer friendly assay to characterize the different PCV2 strains at herd level and to tailor the therapy and vaccination protocols accordingly.

### BRIEF DESCRIPTION OF THE DRAWINGS

Table 1. Origin of porcine circovirus type 2 (PCV2) strains used in this study.

Table 2. Isotype and IPMA antibody titres of hybridoma supernatants.
* IPMA antibody titres of a hybridoma supernatant were expressed as the reciprocal of the last dilution that resulted in a positive reaction.
** MAbs 31D5, 48B5, 59C6 and 108E8 stained 2 different populations of infected cells in strain 1206. IPMA Ab titres for the first population (approximately 99% of the infected cells, at the left of the dash) were comparable to those of the genotype 1 strains 48285, VC2002 and 1147.
IPMA Ab titres for the second population (approximately 1% of the infected cells, at the right of the dash) were comparable to those of the genotype 2 strains 1010, 1121 and 1103.

FIG. 1 Western blotting analysis of Stoon-1010 and mock inoculated PK-15 cells. Odd numbers represent PCV2 inoculated cell lysates, even numbers show mock inoculated lysates. Lanes 1 & 2: mAb F190 (positive control) Lanes 3 & 4: mAb 31D5. Lanes 5 & 6: mAb 38C1. Lanes 7& 8: mAb 108E8. All 4 mAbs reacted specifically with a 28 kDa protein (arrowhead).

Table 3. Neutralizing activity of hybridoma supernatants.
The neutralizing activity of a hybridoma supernatant was expressed as the percentage of reduction in the number of infected cells in comparison with medium. A mean neutralizing activity of 30% or more was considered as neutralization.

FIG. 2 ORF2 amino acid alignment of the PCV2 strains used in this study. Consensus key: *—single, fully conserved residue; :—conservation of strong groups; .—conservation of weak groups; —no consensus (bold)

Table 4. ORF2 amino acid identity within PCV2 strains used in this study. The percentage amino acid identity is given. This is the result of pairwise alignments of the ORF2 proteins. In bold, percentage identity between the genotype 1 strains; in bold and italics, percentage identity between the genotype 2 strains; underlined, percentage identity between the VC2002-k2 strain and other strains.

FIG. 3 Unrooted phylogenetic tree constructed using the NJ method. The percentage confidence is indicated on the branches. This tree was based on the ORF2 protein sequences of the PCV2 strains that were used in the present study (strain names in parentheses), one PCV1 sequence (outgroup) and 20 PCV2 sequences that were obtained from Olvera et al. (2007). These sequences are listed in Table 5.
* No NCBI protein accession number available for the ORF2 protein, so the GenBank nucleotide sequence was used.

Table 5 Name, phylogenetic cluster (according to Olvera et al., 2007) and origin of the sequences used in FIG. 3.

FIG. 4 Provides the nucleic acid (SEQ ID No 5) and the amino acid sequence (SEQ ID No 6) of the ORF encoding for the capsid protein of PCV2 strain 1206

FIG. 5 Provides the nucleic acid (SEQ ID No 7) and the amino acid sequence (SEQ ID No 8) of the ORF encoding for the capsid protein of PCV2 strain VC2002-k39

Provides the nucleic acid (SEQ ID No 9) and the amino acid sequence (SEQ ID No 10) of the ORF encoding for the capsid protein of PCV2 strain VC2002-k2

### DESCRIPTION OF THE INVENTION

This invention is based on the characterization of immunogenic regions in the capsid protein of the porcine circovirus that are associated with the antigenic differences between, hereinafter also referred to as the serotypes between the porcine circoviral strains. In a particular embodiment of the present invention, these immunogenic regions are associated with the differences in genotype (including differences in geographic regions) and the differences in pathogenicity (including differences in clinical representation).

The serotype, i.e. antigenic representation of a PCV2 strain may vary with the animal from which they were isolated or with the clinical stage/presentation from which they were isolated. Using mAbs immunospecific for the aforementioned immunogenic regions, it was demonstrated that serotypes between PCV2 strains, i.e. a difference in genotype (including differences in geographic origin) or a difference in pathogenicity (including differences in clinical representation could be determined.

It is accordingly a first objective of the present invention to provide both the isolated nucleic acid and amino acid sequences encoding for the immunogenic variant capsid proteins of the PCV2 strain, in particular encoding for the immunogenic variant capsid proteins of the PCV2 strains 1206 (SEQ ID No 6) and VC2002 (SEQ ID Nos 8 & 10) as well as immunogenic fragments thereof. The immunogenic fragments comprising at least one epitope selected from the polypeptides;
YTVKRTTVTTPSWAV (AA 55-69 of the Stoon-1010 variant GenBank Accession No AF055392),
GGTNKISIPFEYY (AA 84-95 of the Stoon-1010 variant Genbank Accession No AF055392),
AFENSKYDQDY (AA 201-211 of the Stoon-1010 variant GenBank Accession No AF055392),
DNFYTKATALTYD (AA 127-139 of the Stoon-1010 variant GenBank Accession No AF055392) and
RLQTSGNVDHV (AA 186-196 of the Stoon-1010 variant GenBank Accession No AF055392) or
variants thereof that have at least 70%, 80%, 85%, 90%, 95%, 96%, 97%, 98%, 99% identity to said polypeptides.

In a particular embodiment the immunogenic variants of the capsid proteins of the PCV2 strain or the variants of the epitopes mentioned above, are characterized in that they comprise at least one of the amino acid substitutions selected from the group consisting of;
K63T (lysine instead of threonine at position 63 when numbered in accordance with the amino acid sequence of strain Stoon-1010 Genbank Accession No AF055392)
R63T (arginine instead of threonine at position 63 when numbered in accordance with the amino acid sequence of strain Stoon-1010 Genbank Accession No AF055392)
P88K (proline instead of lysine at position 88 when numbered in accordance with the amino acid sequence of strain Stoon-1010 Genbank Accession No AF055392)
R89I (arginine instead of isoleucine at position 89 when numbered in accordance with the amino acid sequence of strain Stoon-1010 Genbank Accession No AF055392)
I206K (isoleucine instead of lysine at position 206 when numbered in accordance with the amino acid sequence of strain Stoon-1010 Genbank Accession No AF055392)
P131T (proline instead of a threonine at position 131 when numbered in accordance with the amino acid sequence of strain Stoon-1010 Genbank Accession No AF055392)
R191G (arginine instead of a glycine at position 191 when numbered in accordance with the amino acid sequence of strain Stoon-1010 Genbank Accession No AF055392)

As provide in more detail in the examples hereinafter it has particularly been found that differences in PCV2 genotypes are based on the immunogenic variant capsid proteins specified above or immunogenic fragments thereof comprising at least one epitope selected from the polypeptides;
YTVKRTTVTTPSWAV (AA 55-69 of the Stoon-1010 variant GenBank Accession No AF055392),
GGTNKISIPFEYY (AA 84-95 of the Stoon-1010 variant Genbank Accession No AF055392), and
AFENSKYDQDY (AA 201-211 of the Stoon-1010 variant GenBank Accession No AF055392), or
   variants thereof that have at least 70%, 80%, 85%, 90%, 95%, 96%, 97%, 98%, 99% identity to said polypeptides.

In said embodiment (differences in genotype) the immunogenic variants of the capsid proteins of the PCV2 strain or the variants of the epitopes mentioned above, are characterized in that they comprise at least one of the amino acid substitutions selected from the group consisting of;
K63T (lysine instead of threonine at position 63 when numbered in accordance with the amino acid sequence of strain Stoon-1010 Genbank Accession No AF055392);
R63T (arginine instead of threonine at position 63 when numbered in accordance with the amino acid sequence of strain Stoon-1010 Genbank Accession No AF055392);
P88K (proline instead of lysine at position 88 when numbered in accordance with the amino acid sequence of strain Stoon-1010 Genbank Accession No AF055392);
R89I (arginine instead of isoleucine at position 89 when numbered in accordance with the amino acid sequence of strain Stoon-1010 Genbank Accession No AF055392); and
I206K (isoleucine instead of lysine at position 206 when numbered in accordance with the amino acid sequence of strain Stoon-1010 Genbank Accession No AF055392).

For the differences in pathogenicity between the PCV2 strains, it has been found that said differences are based on the immunogenic variant capsid proteins specified above or immunogenic fragments thereof comprising at least one epitope selected from the polypeptides;
DNFYTKATALTYD (AA 127-139 of the Stoon-1010 variant GenBank Accession No AF055392) and
RLQTSGNVDHV (AA 186-196 of the Stoon-1010 variant GenBank Accession No AF055392) or
   variants thereof that have at least 70%, 80%, 85%, 90%, 95%, 96%, 97%, 98%, 99% identity to said polypeptides.

In said embodiment (differences in pathogenicity) the immunogenic variants of the capsid proteins of the PCV2 strain or the variants of the epitopes mentioned above, are characterized in that they comprise at least one of the amino acid substitutions selected from the group consisting of;
P131T (proline instead of a threonine at position 131 when numbered in accordance with the amino acid sequence of strain Stoon-1010 Genbank Accession No AF055392); and
R191G (arginine instead of a glycine at position 191 when numbered in accordance with the amino acid sequence of strain Stoon-1010 Genbank Accession No AF055392).

The term ‘immunogenic’ as used herein, i.e. ‘the immunogenic variants’ and ‘immunogenic fragments’, refer to the capability of said molecules to elicit an immune response in an animal, in particular in a mammal, more in particular in a pig. The immune response may be humoral, cellular, or a combination of both.

Thus, in one aspect the present invention provides an isolated polypeptide selected from the group consisting of;
a) a polypeptide comprising SEQ ID No 6, 8 or 10;
b) a polypeptide comprising at least one, in particular two or three epitopes selected from the polypeptides; YTVKRTTVTTPSWAV, GGTNKISIPFEY, AFENSKYDQDY, DNFYTKATALTYD and RLQTSGNVDHV;
c) a polypeptide encoding a PCV2 capsid protein comprising at least one, in particular two, more in particular three, even more in particular four amino acid substitutions selected from the group consisting of K63T, R63T, P88K, R89I, I206K, P131T and R191G;
d) or a polypeptide comprising at least one epitope that has at least 70%, 80%, 85%, 90%, 95%, 96%, 97%, 98%, 99% identity to the polypeptides of b).

In a further embodiment, the present invention provides an isolated nucleic acid molecule encoding the aforementioned isolated polypeptides. In one aspect said isolated nucleic acid molecule comprises at least one nucleic acid sequence selected from the group consisting of;
TATACTGTCAAGCGTACCACAGTCACAACGCCCTCCTGGGCGGTG (encoding for AA 55-69 of the capsid protein of the Stoon-1010 variant GenBank Accession No AF055392),
GGGGGGACCAACAAAATCTCTATACCCTTTGAATAC (encoding for AA 84-95 of the capsid protein of the Stoon-1010 variant GenBank Accession No AF055392),
GCGTTCGAAAACAGTAAATACGACCAGGACTAC (encoding for AA 201-211 of the capsid protein of the Stoon-1010 variant GenBank Accession No AF055392),
GATAACTTTGTAACAAAGGCCACAGCCCTAACCTATGAC (encoding for AA 127-139 of the capsid protein of the Stoon-1010 variant GenBank Accession No AF055392), and
AGACTACAAACCTCTGGAAATGTGGACCACGTA (encoding for AA 186-196 of the capsid protein of the Stoon-1010 variant GenBank Accession No AF055392).

In a specific embodiment said nucleic acid molecule is selected from the group consisting of;
a) a nucleic acid molecule which is at least 99% identical to SEQ ID No 5; in particular consists of SEQ ID No 5;
b) a nucleic acid molecule which is at least 99% identical to SEQ ID No 7; in particular consists of SEQ ID No 7;
c) a nucleic acid molecule which is at least 95%, 96%, 97%, 98% or 99% identical to SEQ ID No 9; in particular consists of SEQ ID No 9; and
d) the complementary sequence to any one of the above.

The invention also provides nucleic acids that are fragments of the nucleic acids encoding a polypeptide of the invention. In one aspect, the invention provides nucleic acids primers or probes which consist essentially of from 15 to 50, for example from 15 to 35, 18 to 35, 15 to 24, 18 to 30, 18 to 21 or 21 to 24 nucleotides of a sequence encoding a polypeptide of the invention or its complement.

The term “consist essentially of” refers to nucleic acids which do not include any additional 5′ or 3′ nucleic acid sequences. In a further aspect of the invention, nucleic acids of the invention which consist essentially of from 15 to 30 nucleotides as defined above may however be linked at the 3′ but preferably 5′ end to short (e.g from 4 to 15, such as from 4 to 10 nucleotides) additional sequences to which they are not naturally linked. Such additional sequences are preferably linkers which comprise a restriction enzyme recognition site to facilitate cloning when the nucleic acid of the invention is used for example as a PCR primer.

Primers and probes of the invention are desirably capable of selectively hybridising to nucleic acids encoding the polypeptides of the invention. By “selective”, it is meant selective with respect to sequences encoding other PCV2 capsid proteins. The ability of the sequence to hybridize selectively may be determined by experiment or calculated.

For example, one way to calculate Tm of a primer is by reference to the formula for calculating the Tm of primers to a homologous target sequence. This formula is Tm(° C.)=2(A+T)+4(G+C)-5. This will provide the Tm under conditions of 3×SSC and 0.1% SDS (where SSC is 0.15M NaCl, 0.015M sodium citrate, pH 7). This formula is generally suitable for primers of up to about 50 nucleotides in length. In the present invention, this formula may be used as an algorithm to calculate a nominal Tm of a primer for a specified sequence derived from a sequence encoding a polypeptide of the invention. The Tm may be compared to a calculated Tm for GPCR sequences of humans and rats, based upon the maximum number of matches to any part of these other sequences.

Suitable conditions for a primer to hybridize to a target sequence may also be measured experimentally. Suitable experimental conditions comprise hybridising a candidate primer to both nucleic acid encoding a polypeptide of the invention and nucleic acid encoding other PCV2 capsid proteins on a solid support under low stringency hybridising conditions (e.g. 6×SSC at 55° C.), washing at reduced SSC and/or higher temperature, for example at 0.2×SSC at 45° C., and increasing the hybridisation temperature incrementally to determine hybridisation conditions which allow the primer to hybridize to nucleic acid encoding a polypeptide of the invention but not other PCV2 capsid protein encoding nucleic acids.

Nucleic acids of the invention, particularly probes, may carry a revealing label. Suitable labels include radioisotopes such as ³²P or ³⁵S, fluorescent labels, enzyme labels, or other protein labels such as biotin. Such labels may be added to polynucleotides or primers of the invention and may be detected using by techniques known per se.

In a particular embodiment the primers or probes selectively hybridize to the isolated nucleic acid sequences that encode for the epitopes having a polypeptide sequence selected from YTVKRTTVTTPSWAV, GGTNKISIPFEY, AFENSKYDQDY, DNFYTKATALTYD and RLQTSGNVDHV or variants thereof which have at least 70%, 80%, 85%, 90%, 95%, 96%, 97%, 98%, 99% identity to said polypeptides.

In a more particular embodiment the primers or probes selectively hybridize to the isolated nucleic acids selected from;
TATACTGTCAAGCGTACCACAGTCACAACGCCCTCCTGGGCGGTG (encoding for AA 55-69 of the capsid protein of the Stoon-1010 variant GenBank Accession No AF055392),
GGGGGGACCAACAAAATCTCTATACCCTTTGAATAC (encoding for AA 84-95 of the capsid protein of the Stoon-1010 variant GenBank Accession No AF055392),
GCGTTCGAAAACAGTAAATACGACCAGGACTAC (encoding for AA 201-211 of the capsid protein of the Stoon-1010 variant GenBank Accession No AF055392),
GATAACTTTGTAACAAAGGCCACAGCCCTAACCTATGAC (encoding for AA 127-139 of the capsid protein of the Stoon-1010 variant GenBank Accession No AF055392), and
AGACTACAAACCTCTGGAAATGTGGACCACGTA (encoding for AA 186-196 of the capsid protein of the Stoon-1010 variant GenBank Accession No AF055392).

In one embodiment, the present invention relates to fusion proteins, comprising the aforementioned PCV2 capsid proteins, fragments or the epitopes thereof and a heterologous protein or part of a protein acting as a fusion partner. The proteins of the present invention and the fusion partner may be chemically conjugated, but are preferably expressed as recombinant fusion proteins in a heterologous expression system. The fusion partner can either be an immunological fusion partner that may assist in providing T helper epitopes, or act as an expression enhancer. Thus the immunological fusion protein may act through a bystander helper effect linked to the secretion of activation signals by a large number of T-cells specific to the foreign protein or peptide, thereby enhancing the induction of immunity to the PCV2 capsid protein.

It is accordingly an object of the present invention to provide the use of a polypeptide according to the present invention as an immunogen or otherwise in obtaining specific antibodies. Antibodies are useful in purification and other manipulation of polypeptides, diagnostic screening and therapeutic contexts.

Thus in a further aspect, the present invention provides antibodies, that specifically bind with the immunogenic regions in the capsid protein of the porcine circovirus as identified by the present invention, i.e. with one or more of the polypeptides selected from the group consisting of;
a. a polypeptide comprising SEQ ID No 6, 8 or 10;
b. a polypeptide comprising at least one, in particular two or three of the epitopes selected from the polypeptides; YTVKRTTVTTPSWAV, GGTNKISIPFEY, AFENSKYDQDY, DNFYTKATALTYD and RLQTSGNVDHV;
c. a polypeptide encoding a PCV2 capsid protein comprising at least one, in particular two, more in particular three, even more in particular 4 amino acid substitutions selected from the group consisting of K63T, R63T, P88K, R891, I206K, P131T and R191G; or
d. a polypeptide comprising at least one epitope that has at least 70%, 80%, 85%, 90%, 95%, 96%, 97%, 98%, 99% identity to the polypeptides of b).

In one embodiment said immunogenic region may consist of a three-dimensional epitope recognised by said antibodies. In another embodiment said immunogenic region may consist of a linear epitope recognised by the antibodies. In a particular embodiment the immunogenic region comprising the immunogenic capsid PCV2 proteins, and one or more of the immunogenic fragments or polypeptides as defined hereinbefore. It is accordingly an object of the present invention to provide the immunogenic regions recognized by said antibodies, as well as the therapeutic and diagnostic use thereof.

Said antibodies may be polyclonal or monoclonal antibodies, that can be obtained using known techniques, and in a particular embodiment consist of monoclonal antibodies, more in particular of the monoclonal antibodies 13H4, 31D5, 48B5, 59C6, 108E8, 38C1 and 21C12. The antibodies 13H4, 31D5, 48B5, 59C6 and 108E8 were deposited at the Belgian Co-ordinated Collection of Micro-organisms with the references 13H4, 31D5, 48B5, 59C6 and 108E8 on 17 Aug. 2007 and received the respective depositnumbers LMBP 6586CB, LMBP 6587CB, LMBP 6588CB, LMBP 6589CB, and LMBP 6590CB. The antibodies 21C12 were deposited at the Belgian Co-ordinated Collection of Micro-organisms with the references 21C12 on 21 Apr. 2008 and received the respective depositnumbers LMBP 6659CB. The antibodies 38C1 were deposited at the Belgian Co-ordinated Collection of Micro-organisms with the references 38C1 on 2 Jun. 2008 and received the respective depositnumbers LMBP 6660CB.

As provided hereinafter, the monoclonal antibodies of the present invention include those produced by hybridomas, as well as the recombinant antibodies obtainable thereof.

Monoclonal antibodies produced by hybridomas are obtained using art know techniques. It typically comprises immunizing an animal using PCV2 capsid protein, in particular using the PCV2 capsid protein of PCV2 strain Stoon-1010 as a sensitizing antigen to obtain an immune cell, such as a splenocyte or lymph node cell that is isolated and subsequently fused to an appropriate immortalized cell such as a myeloma cell line. The cell fusion of the immune cell to the myeloma cell is essentially done using art known procedures, such as for example provided in the examples hereinafter or the method of Galfre & Milstein et al. (Galfre G. and Milstein C. Methods Enzymol. (1981) 73, 1-46). Finally, a conventional limiting dilution method is carried out for screening and single cloning of a hybridoma producing the intended antibody.

The recombinant monoclonal antibodies according to the invention can be generated using art known procedures, comprising cloning the gene of the antibody from the hybridoma, integrating the gene in an appropriate vector, introducing the gene into a host, and allowing the recombinant antibody to be produced by the host.

The gene of the recombinant antibody may be expressed by transforming the host with DNA encoding the Heavy chain (H chain) and DNA encoding the Light chain (L chain) of said antibody. In a further aspect the present invention provides chimeric antibodies that are obtained by combining the DNA encoding the Variable region of the antibodies according to the invention with the DNA encoding the desired Constant region to obtain chimeric antibodies.

It is a further objective of the present invention to provide the use of the antibodies according to the invention in a method to identify antigenic differences, i.e. serotypes between PCV2 strains with a different genotype and originating from different clinical presentations. It is accordingly an object of the present invention to provide kits comprising said antibodies and all elements needed to perform the desired diagnostic method. Examples of different diagnostic methods and elements needed therewith, are provided in more detail hereinafter. In one embodiment the methods are performed using of immunoassays and the corresponding immunoassay kits comprise the antibodies according to the invention and all elements needed to perform the desired immunoassay, including without limitation, reagents (for example, an enzyme, a radioisotope, a fluorescent reagent, a luminescent reagent, a chemiluminescent reagent, etc.); a solid surface, such as beads, to which an antibody of the present invention is affixed; buffers; positive and negative controls; and other suitable components. In a particular embodiment the immunoassay kit is an ELISA kit.

In the methods and kits to determine differences in genotype between PCV2 strains, said methods and kits comprising the use of one or more antibodies specific for or selectively binding to one or more of the polypeptides selected from the group consisting of;
a. a polypeptide comprising SEQ ID No 6, 8 or 10;
b. a polypeptide comprising at least one, in particular two or three epitopes selected from the polypeptides; YTVKRTTVTTPSWAV, GGTNKISIPFEY and AFENSKYDQDY;
c. a polypeptide encoding a PCV2 capsid protein comprising at least one, in particular two, more in particular three, even more in particular 4 amino acid substitutions selected from the group consisting of K63T, R63T, P88K, R891 and I206K; and
d. a polypeptide comprising at least one epitope that has at least 70%, 80%, 85%, 90%, 95%, 96%, 97%, 98%, 99% identity to the polypeptides of b)

In a particular embodiment of the methods and kits to genotype the different PCV2 strains, the antibodies are selected from the group consisting of the monoclonal antibodies 31D5, 48B5, 59C6 and 108E8 (supra).

In the methods and kits to determine differences in pathogenicity between PCV2 strains, said methods and kits comprising the use of one or more antibodies specific for or selectively binding to one or more of the polypeptides selected from the group consisting of;
a. a polypeptide comprising SEQ ID No 6, 8 or 10;
b. a polypeptide comprising at least one, in particular two of the epitopes selected from the polypeptides; DNFYTKATALTYD and RLQTSGNVDHV;
c. a polypeptide encoding a PCV2 capsid protein comprising at least one, in particular both of the amino acid substitutions selected from the group consisting of P131T and R191G; and
d. a polypeptide comprising at least one epitope that has at least 70%, 80%, 85%, 90%, 95%, 96%, 97%, 98%, 99% identity to the polypeptides of b)

In a particular embodiment of the methods and kits to determine the pathogenicity of the different PCV2 strains the antibody consists of the monoclonal antibody 13H4.

Thus, the invention relates to methods of diagnosing and/or predicting antigenic differences between PCV2 strains in an animal, by measuring the expression of a PCV2 capsid protein in said animal. For example an increased level of one or more of the polypeptides according to the invention, in particular encoding the PCV2 capsid protein variants (e.g., SEQ ID No 6, 8 or 10).

Diagnostic methods for the detection of PCV2 capsid protein nucleic acid molecules, in animal samples or other appropriate cell sources, may involve the amplification of specific gene sequences, e.g., by PCR (See Mullis, K. B., 1987, U.S. Pat. No. 4,683,202), followed by the analysis of the amplified molecules using techniques well known to those of skill in the art.

Alternatively, the diagnosis of antigenic differences of PCV2 strains pertain to the detection of the PCV2 capsid protein variants, fragments or epitopes as defined hereinbefore. Detection of the polypeptide according to the invention may be by any method known in the art.

The tissue or cell type to be analyzed generally includes those which are known, or suspected, to express the PCV2 capsid protein, such as, for example PK-15 cells, SK-cells, ST-cells, 3D4/31-cells, porcine PBMC (peripheral blood mononuclear cells), porcine alveolar macrophages, porcine lymphoïd tissues such as lymph nodes, spleen, tonsils and thymus and porcine non-lymphoid tissues such as lungs, liver, kidney, heart and intestines.

Preferred diagnostic methods for the detection of antigenic difference in PCV2 strains may involve, for example, immunoassays wherein the polypeptides according to the invention, are detected by their interaction with selective antibodies. For example, antibodies, or fragments of antibodies as provided hereinbefore, may be used to quantitatively or qualitatively detect the presence of the polypeptides according to the invention.

Immunoassays for PCV2 capsid proteins, fragments or epitopes thereof, will typically comprise contacting a sample, such as a biological fluid, tissue or a tissue extract, freshly harvested cells, or lysates of cells which have been incubated in cell culture, in the presence of an antibody that specifically or selectively binds to the polypeptides of the invention, e.g., a detectably labeled antibody capable of identifying the polypeptides of the present invention, and detecting the bound antibody by any of a number of techniques well-known in the art (e.g., Western blot, ELISA, FACS). The biological sample may be brought in contact with and immobilized onto a solid phase support or carrier such as nitrocellulose, or other solid support that is capable of immobilizing cells, cell particles or soluble proteins. The support is washed with suitable buffers followed by treatment with a blocking agent and the labeled antibody that selectively or specifically binds to a PCV2 capsid protein encoded polypeptide. The solid phase support is washed with buffer a second time to remove unbound antibody. The amount of bound label on a solid support may be detected by conventional means. Alternatively, the antibody that selectively or specifically binds to a PCV2 capsid protein encoded polypeptide is immobilized, and the biological sample comprising a polypeptide according to the invention incubated therewith.

The present invention further provides immunological techniques that can be useful in the detection of PCV2 strains or previous PCV2 infections, i.e. using the monoclonal antibodies of the present invention to detect PCV2 specific antibodies in a sample. Briefly, sera or other body fluids from the subject is reacted with the antigen bound to a substrate (e.g. an ELISA 96-well plate). Excess sera is thoroughly washed away. A labeled (enzyme-linked, fluorescent, radioactive, etc.) monoclonal antibody is then reacted with the previously reacted antigen-serum antibody complex. The amount of inhibition of monoclonal antibody binding is measured relative to a control (no patient serum antibody). The degree of monoclonal antibody inhibition is a very specific test for a particular variety or strain since it is based on monoclonal antibody binding specificity. This competitive ELISA is in particular useful in serotyping PCV2 infections in a convenient and cost-effective way.

Alternatively, micro-agglutination test can also be used to detect the presence of antibodies for the PCV2 strain variants of the present invention in a sample. Briefly, a solid phase supports or carriers are coated with the antigen and mixed with a sample from the subject, such that antibodies in the tissue or body fluids that are specifically reactive with the antigen crosslink with the antigen, causing agglutination. The agglutinated antigen-antibody complexes form a precipitate, visible with the naked eye or by spectrophotometer. In a modification of the above test, antibodies specifically reactive with the antigen can be bound to the beads and antigen in the tissue or body fluid thereby detected.

In addition, as in a typical sandwich assay, the antibody can be bound to a substrate and reacted with the antigen. Thereafter, a secondary labeled antibody is bound to epitopes not recognized by the first antibody and the secondary antibody is detected. Since the present invention provides PCV2 antigen for the detection of infectious PCV2 or previous PCV2 infection other serological methods such as flow cytometry and immunoprecipitation can also be used as detection methods.

In the diagnostic methods taught herein, the antigen can be bound to a substrate and contacted by a biological fluid sample such as serum, urine, saliva, feces or gastric juice. This sample can be taken directly from the patient or in a partially purified form. In this manner, antibodies specific for the antigen (the primary antibody) will specifically react with the bound antigen. Thereafter, a secondary antibody bound to, or labeled with, a detectable moiety can be added to enhance the detection of the primary antibody. Generally, the secondary antibody or other ligand which is reactive, either specifically with a different epitope of the antigen or nonspecifically with the ligand or reacted antibody, will be selected for its ability to react with multiple sites on the primary antibody. Thus, for example, several molecules of the secondary antibody can react with each primary antibody, making the primary antibody more detectable.

By “solid phase support or carrier” is intended any support capable of binding an antigen or an antibody. Well-known supports or carriers include glass, polystyrene, polypropylene, polyethylene, dextran, nylon, nitrocellulose, natural and modified celluloses, polyacrylamides, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The anti-PCV2 capsid protein antibody can be detectably labeled by linking the same to an enzyme and using the labeled antibody in an enzyme immunoassay (EIA) (Voller, A., “The Enzyme Linked Immunosorbent Assay(ELISA)”, 1978, Diagnostic Horizons 2:1, Microbiological Associates QuarterlyPublication, Walkersville, Md.); Voller, A. et al., 1978, J. Clin. Pathol. 31: 507-520; Butler, J. E., 1981, Meth. Enzymol. 73: 482; Maggio, E. (ed.), 1980, EnzymeImmunoassay, CRC Press, Boca Raton, Fla.; Ishikawa, E. et al., (eds.), 1981, Enzyme Immunoassay, Kgaku Shoin, Tokyo). The enzyme that is bound to the antibody will react with an appropriate labeled substrate, preferably a fluoresceinisothiocyanate, rhodamine, phycoerythrin, phycocyanin, allophycocyanin, o-phthaldehyde and fluorescamine labeled substrate.

The antibody can also be detectably labeled using fluorescence emitting metals such as ¹⁵²Eu, or others of the lanthanide series. These metals are attached to an antibody using such metal chelating groups as diethylenetriaminepentacetic acid (DTPA) or ethylenediaminetetraacetic acid (EDTA). Fluorochromes typically used are Fluorescein, Texas Red or other fluorochromes such as the Alexa Fluor series.

The antibody can also be detectably labeled by coupling it to a chemiluminescent compound. The presence of the chemiluminescent-tagged antibody is detected by luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of a chemiluminescent reaction. The presence of a bioluminescent protein is determined by detecting luminescence. Important bioluminescent compounds for purposes of labeling are luciferin, luciferase and aequorin.

Hence, in one embodiment the present invention provides a method to identify genotypic differences between PCV2 strains said method comprising contacting a sample, such as a biological fluid, tissue or a tissue extract, freshly harvested cells, or lysates of cells which have been incubated in cell culture, with an antibody, e.g. a detectably labeled antibody that specifically or selectively binds to a polypeptide selected from the group consisting of;
b) a polypeptide comprising SEQ ID No 6, 8 or 10;
c) a polypeptide comprising at least one, in particular two or three of the epitopes selected from the polypeptides; YTVKRTTVTTPSWAV, GGTNKISIPFEY, AFENSKYDQDY;
d) a polypeptide encoding a PCV2 capsid protein comprising at least one, in particular two, more in particular three, even more in particular 4 amino acid substitutions selected from the group consisting of K63T, R63T, P88K, R891, I206K; or
e) a polypeptide comprising at least one epitope that has at least 70%, 80%, 85%, 90%, 95%, 96%, 97%, 98%, 99% identity to the polypeptides of b); detecting the binding of the antibody to any one of said polypeptides; and
   detecting the binding of the antibody to any one of said polypeptides

In one embodiment of the aforementioned method, the antibody is a monoclonal antibody, in particular a monoclonal antibody selected from the group consisting of 31D5, 48B5, 59C6 and 108E8.

In analogy, in another embodiment the present invention provides a method to identify differences in pathogenicity (including differences in clinical presentation) between PCV2 strains said method comprising contacting a sample, such as a biological fluid, tissue or a tissue extract, freshly harvested cells, or lysates of cells which have been incubated in cell culture, with an antibody, e.g. a detectably labeled antibody that specifically or selectively binds to a polypeptide selected from the group consisting of;
a) a polypeptide comprising SEQ ID No 6, 8 or 10;
b) a polypeptide comprising at least one, in particular two of the epitopes selected from the polypeptides; DNFYTKATALTYD and RLQTSGNVDHV;
c) a polypeptide encoding a PCV2 capsid protein comprising at least one, in particular both of the amino acid substitutions selected from the group consisting of P131T and R191G;
d) or a polypeptide comprising at least one epitope that has at least 70%, 80%, 85%, 90%, 95%, 96%, 97%, 98%, 99% identity to the polypeptides of b); and
   detecting the binding of the antibody to any one of said polypeptides

The specific antibody may be any one of the antibodies described hereinbefore, in particular one of the monoclonal antibodies described herein but, often an antibody-derivative is used which preferably is selected from the group of antibody fragments, conjugates or homologues, but also complexes and adsorbates known to the skilled artisan.

In one embodiment of the aforementioned method, the antibody is a monoclonal antibody, in particular said monoclonal antibody is 13H4.

In a further object, the present invention provides the use of immunogenic PCV2 capsid proteins, immunogenic fragments or polypeptides as defined hereinbefore or/and the monoclonal antibodies of the present invention in antigenic typing a PCV2 infection or a previous PCV2 infection.

In one embodiment the present invention provides a method to determine antibody titres in a sample, said method comprising contacting the immunogenic PCV2 capsid proteins, immunogenic fragments or polypeptides with a fluid sample such as serum; and determine the presence of an antigen-serum antibody complex using a monoclonal antibody of the present invention.

Given the characterization of monoclonal antibodies capable to identify antigenic differences between PCV2 strains, it is also an object of the present invention to provide the use of said monoclonal antibodies in isolating said PCV2 strains, i.e. PCV2 antigenic subtypes (PCV2 serotypes with antigenic differences, thus recognized by different Mabs), using art known procedures.

The thus isolated PCV2 strains/antigenic subtypes can be used in diagnosis or vaccine production. Hence in one embodiment the present invention provides a vaccine comprising a PCV2 strain/antigenic subtypes obtained using the aforementioned isolation.

Killed (inactivated) or live vaccines can be produced. To make a live vaccine, a viral isolate, or an attenuated or mutated variant thereof, is grown in cell culture. The virus is harvested according to methods well known in the art. The virus may then be concentrated, frozen, and stored at −70° C., or freeze-dried and stored at 4° C. Prior to vaccination the virus is mixed at an appropriate dosage, (which is from about 10 to 10⁸ tissue culture infectious doses per ml), with a pharmaceutically acceptable carrier such as a saline solution, and optionally an adjuvant.

The vaccine produced might also comprise an inactivated or killed vaccine comprising a PCV2 strain obtained by the methods of the invention. The inactivated vaccine is made by methods well known in the art. For example, once the virus is propagated to high titers, it would be readily apparent by those skilled in the art that the virus antigenic mass could be obtained by methods well known in the art. For example, the virus antigenic mass may be obtained by dilution, concentration, or extraction. All of these methods have been employed to obtain appropriate viral antigenic mass to produce vaccines. The virus is then inactivated by treatment with formalin, betapropriolactone (BPL), binary ethyleneimine (BEI), or other methods known to those skilled in the art. The inactivated virus is then mixed with a pharmaceutically acceptable carrier such as a saline solution, and optionally an adjuvant. Examples of adjuvants include, but not limited to, aluminum hydroxide, oil-in-water and water-in-oil emulsions, AMPHIGEN, saponins such as QuilA, and polypeptide adjuvants including interleukins, interferons, and other cytokines.

Inactivation by formalin is performed by mixing the viral suspension with 37% formaldehyde to a final formaldehyde concentration of 0.05%. The virus-formaldehyde mixture is mixed by constant stirring for approximately 24 hours at room temperature. The inactivated virus mixture is then tested for residual live virus by assaying for growth on a suitable cell line.

Inactivation by BEI is performed by mixing the viral suspension of the present invention with 0.1 M BEI (2-bromo-ethylamine in 0.175 N NaOH) to a final BEI concentration of 1 mM. The virus-BEI mixture is mixed by constant stirring for approximately 48 hours at room temperature, followed by the addition of 1.0 M sodium thiosulfate to a final concentration of 0.1 mM. Mixing is continued for an additional two hours. The inactivated virus mixture is tested for residual live virus by assaying for growth on a suitable cell line.

The present invention now has as its object to provide the use of a PCV2 capsid protein epitope, mimotope, specific or anti-idiotypic antibody; including a PCV2 strain comprising a PCV2 capsid protein variant or epitope for preparing a medicament which is employed for the prophylactic and/or therapeutic treatment of PCV infection in animals, in particular in swine and piglets.

In one embodiment the PCV2 capsid protein epitope is selected from the group of the peptides or proteins as described herein, in particular comprising a polypeptide selected of;
a) the polypeptides; YTVKRTTVTTPSWAV, GGTNKISIPFEY, AFENSKYDQDY, DNFYTKATALTYD and RLQTSGNVDHV;
b) or a polypeptide that has at least 70%, 80%, 85% 90%, 95%, 96%, 97%, 98%, 99% identity to the polypeptides of a).

In another embodiment variants of the capsid proteins of the PCV2 strain or of the epitopes mentioned above, are characterized in that they comprise at least one, in particular two, more in particular three, even more in particular four amino acid substitutions selected from the group consisting of;
K63T (lysine instead of threonine at position 63 when numbered in accordance with the amino acid sequence of strain Stoon-1010 Genbank Accession No AF055392)
R63T (arginine instead of threonine at position 63 when numbered in accordance with the amino acid sequence of strain Stoon-1010 Genbank Accession No AF055392)
P88K (proline instead of lysine at position 88 when numbered in accordance with the amino acid sequence of strain Stoon-1010 Genbank Accession No AF055392)
R89I (arginine instead of isoleucine at position 89 when numbered in accordance with the amino acid sequence of strain Stoon-1010 Genbank Accession No AF055392)
I206K (isoleucine instead of lysine at position 206 when numbered in accordance with the amino acid sequence of strain Stoon-1010 Genbank Accession No AF055392)
P131T (proline instead of a threonine at position 131 when numbered in accordance with the amino acid sequence of strain Stoon-1010 Genbank Accession No AF055392)
R191G (arginine instead of a glycine at position 191 when numbered in accordance with the amino acid sequence of strain Stoon-1010 Genbank Accession No AF055392)

Accordingly the invention also provides a vaccine comprising;
a PCV2 capsid protein epitope, mimotope, specific or anti-idiotypic antibody according to the invention; or
a PCV2 strain (killed or inactivated) comprising a PCV2 capsid protein variant or epitope according to the invention.

The vaccine used according to the invention advantageously is provided in a suitable formulation. Preferred are such formulations with a pharmaceutically acceptable carrier. This comprises, e.g., auxiliary substances, buffers, salts, preservatives.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### EXAMPLES

The following examples illustrate the invention. Other embodiments will occur to the person skilled in the art in light of these examples.

### Methods

### Viruses

Seven different PK-15 adapted PCV2 strains were used in this study. Their origin and to genotype (Olvera et al., 2007) are shown in Table 1. The replication kinetics of these strains have been documented previously (Meerts et al., 2005a). PCV1 originated from the persistently infected PK-15 cell line ATCC-CCL33.

### Recombinant PCV2 Virus-Like Particles

PCV2 virus-like particles (VLPs) were obtained by infecting Spodoptera frugiperda 9 (Sf9) insect cells with a baculovirus recombinant P054 expressing the ORF2 of PCV2 strain Stoon-1010. Purification of VLPs was performed in a caesium chloride gradient as described by Nawagitgul et al. (2000).

### Cells

PCV negative PK-15 cells and the persistently PCV1 infected PK-15 cell line ATCC-CCL33 were grown in minimal essential medium (MEM) containing Earle's salts (Gibco, Grand Island, USA), supplemented with 5% or 10% foetal bovine serum (FBS), 0.3 mg ml⁻¹ glutamine, 100 U ml⁻¹ penicillin, 0.1 mg ml⁻¹ streptomycin and 0.1 mg ml⁻¹ kanamycin. Cell cultures were maintained at 37° C. in the presence of 5% CO2.

### Mouse Immunisation

Before immunisation, mice were made immuno-tolerant to PK-15 cells as described by Matthew and Sandrock (1987). Four 6-weeks-old female Balb/c mice were injected intraperitoneally (IP) with 1.5×10⁷ PCV negative PK-15 cells in a volume of 300 μl phosphate-buffered saline (PBS). Ten minutes, 24 hours and 48 hours later, cyclophosphamide (Sigma, Bornem, Belgium) was injected IP at a dose of 100 mg kg⁻¹ body weight in a total volume of 500 μl PBS. Three and six weeks later, injections with PK-15 cells and cyclophosphamide were repeated. Two weeks after the last treatment, 2.25×10⁷ Stoon-1010 inoculated PK-15 cells were injected IP in a volume of 300 μl PBS mixed with an equal amount of complete Freund's adjuvant (Sigma). At this time point and two weeks later, sera of mice were collected. Three weeks after the inoculation with PCV2, one mouse received an IP injection with 4.5×10⁷ Stoon-1010 inoculated PK-15 cells diluted in 600 μl PBS. Euthanasia was performed 4 days later and the spleen was collected.

### Production and Screening of Hybridomas

Hybridoma cells were produced by fusion of spleen cells with SP 2/0 myeloma cells as described by Galfre and Milstein (1981). The resulting hybridoma cells were maintained in RPMI 1640 (Gibco, Grand Island, USA) supplemented with 10% FBS. PCV2-specific mAbs in supernatant fluids were demonstrated on PCV negative and Stoon-1010 inoculated PK-15 cells by an IPMA adapted from Labarque et al. (2000). After incubation with undiluted supernatant fluids for 1 h at 37° C., cells were washed twice with PBS. Subsequently, a 1:500 dilution of horseradish-peroxidase-labelled goat anti-mouse polyclonal antibodies (Abs) (Dako, Glostrup, Denmark) in PBS were added for 1 h at 37° C. After washing twice in PBS, substrate solution was added and cell cultures were analyzed by light microscopy (Olympus Optical Co., Hamburg, Germany). Selected hybridoma cultures were cloned by limiting dilution.

### Determination of Monoclonal Antibody Class

The isotype of the produced mAbs was determined using a peroxidase-based commercial mouse mAb identification kit (Zymed, San Francisco, USA). This test identifies the IgG1, IgG2a, IgG2b, IgG3, IgA and IgM isotype classes and the κ and λ type of light chains by the use of mono-specific rabbit polyclonal Abs. Supernatant fluids of anti-PRV mAbs 13D12 (IgG1) and 1C11 (IgG2a) (Nauwynck and Pensaert, 1995) and anti-E. coli mAb E7G3 (IgG3) (Tiels et al., 2007) were used as positive controls.

### Indirect Immunofluorescence Staining of Recombinant PCV2 Virus-Like Particles

The VLP staining technique was adapted from Misinzo et al. (2005). Briefly, purified VLPs were diluted 1:100 in PBS, smeared onto microscope slides, air-dried and fixed with 3% (w/v) paraformaldehyde in PBS for 10 min at room temperature. Fixed VLPs were incubated with undiluted hybridoma supernatants for 1 h at 37° C., followed by a 1:500 dilution of FITC-labelled goat anti-mouse Abs (Molecular Probes, Eugene, USA) containing 10% PCV2 negative goat serum (NGS) for 1 h at 37° C. MAb F217 (McNeilly et al., 2001) diluted 1:50 in PBS was used as a positive control. MAbs 13D12 and 1C11 were included as negative controls. A Leica DM/RBE fluorescence microscope (Leica Microsystems GmbH, Heidelberg, Germany) was used for visualisation.

### Western Blot Analysis

Stoon-1010-inoculated and mock-inoculated PCV negative PK-15 cells were harvested by scraping. Cells were pelleted by centrifugation at 15,700×g for 20 min at 4° C. and subsequently lysed for 1 h at 37° C. in TNE (50 mM Tris-HCl [pH 7.4], 150 mM NaCl, 1 mM EDTA) containing 1% NP-40 (Roche, Mannheim, Germany), protease inhibitors (Complete; Roche, Mannheim, Germany) and 0.5% SDS. Cells were centrifuged at 15,700×g for 10 min at 4° C. and resuspended in a non-reducing Laemmli buffer. This mixture was boiled for 5 min and stored at −20° C. until use. Proteins were separated by standard SDS-PAGE and transferred to a PVDF membrane (Amersham Biosciences, England). This membrane was then incubated for 1 h at room temperature in PBS containing 0.1% Tween 20 (PBS-Tween), supplemented with 5% bovine serum albumin (Sigma, Bornem, Belgium).

After washing in PBS-Tween, membranes were incubated overnight at 4° C. with a 1:5 dilution of the mAbs in PBS-Tween. MAb F190 (McNeilly et al., 2001) and biotinylated purified porcine polyclonal Abs, originated from a PCV2 negative SPF pig inoculated with strain 1121 (Pensaert et al., 2004; Meerts et al., 2005a), were used as positive controls. MAbs 13D12 and 1C11 were included as negative controls. Afterwards, a 1:300 dilution of biotinylated polyclonal sheep anti-mouse Abs and a 1:300 solution of a streptavidin-biotinylated-horseradish peroxidase complex (Amersham Biosciences, England) were applied. Membranes were washed twice with PBS-Tween in between the incubations. Antigen-Ab complexes were visualised by an enhanced chemiluminescence assay (Amersham Biosciences, England).

### Reactivity of Monoclonal Antibodies to Different PCV2 Strains

PCV2 strains Stoon-1010, 48285, 1206, VC2002, 1147, 1121 and 1103 were used to make 96-well IPMA plates as described by Labarque et al. (2000). PCV negative PK-15 cells and the persistently PCV1 infected PK-15 cell line were used for control IPMA plates. The staining procedure was similar to the IPMA technique described above. Ten-fold dilutions of hybridoma supernatants were made in PBS and used as primary Abs. IPMA antibody titres of a hybridoma supernatant were expressed as the reciprocal of the last dilution that resulted in a positive reaction. These assays were performed 3 times.

### Sensitive Neutralization Assays

In order to detect the neutralizing activity of the mAbs, a sensitive neutralization assay was adapted from Meerts et al. (2005b). Briefly, 10^{4.3} TCID50 PCV2 in a volume of 200 μl was incubated for 1 h at 37° C. with 200 μl of undiluted hybridoma supernatant. After incubation, this mixture was added to semi-confluent monolayers of PCV negative PK-15 cells in 4 wells of a 96-well plate. After 1 h at 37° C., cell cultures were washed twice in MEM and fresh medium was added. Cell cultures were fixed 36 hours later. At this time point the first replication cycle of PCV2 was completed (Meerts et al., 2005a). PCV2 infected PK-15 cells were stained by an IPMA using porcine polyclonal PCV2-specific Abs, originating from a Stoon-1010 inoculated gnotobiotic pig. The number of infected cells per well was determined by light microscopy. The neutralizing activity of a hybridoma supernatant was expressed as the percentage of reduction in the number of infected cells in comparison with medium. Assays were performed with all 7 strains. Anti-PCV2 mAb F190 was used as a positive control. MAbs 13D12 and 1C11 were used as negative controls. A mAb was considered as neutralizing when its mean neutralizing activity was higher than the mean neutralizing activity+the standard deviation of the negative controls. Sensitive neutralization experiments were performed 3 times.

Sequencing of ORF2 from Strains 1206 and VC2002

The Belgian PCV2 strains 1206 and VC2002 were purified by ultracentrifugation at 180,000×g for 3 h through a 30% sucrose gradient as described by Delputte et al. (2002). A set of PCR primers was designed based on the alignment of the genome sequences of strains Stoon-1010, 48285, 1147, 1121 and 1103. The primer set PCV2-FW (5′ phosphate AGCGCACTTCTTTCGTTTTCAG) (SEQ ID No 1) and PCV2-REV: (5′ phosphate GAATGCGGCCGCTTATCACTTCGTAATGGTTTTTATTATTCA) (SEQ ID No 2) amplifies the complete ORF2. Two internal oligonucleotides were synthesized: CV1 (5′GGGCTGTGGCCTTTGKTAC) (SEQ ID No 3) and CV2 (5′TGTRGACCACGTAGGCCTCG) (SEQ ID No 4).

These internal oligonucleotides were used for sequencing. A 1/200 fraction of proteinaseK treated ultra purified PCV2 virus was used as template in PCR reactions using Platinum Pfx DNA polymerase (Invitrogen, Merelbeke, Belgium) at 60° C. annealing temperature and using the cycling conditions as described by the manufacturer. PCR products (approximately 800 bp) were treated with Exonuclease I and Antarctic Phosphatase (New England Biolabs, Ipswich, USA) and used directly for cycle sequencing with a Big Dye Terminator Cycle sequencing kit V1.1 (Applied Biosystems, Foster City, USA) and PCV2 primers. Cycle sequencing reaction products were purified using ethanol precipitation and separated on an ABI Genetic Analyzer 310 (Applied Biosystems, Foster City, USA). Additionally, PCR products (approximately 800 bp) were gel purified using a QiaQuick gel extraction kit (Qiagen Benelux, Venlo, The Netherlands) and cloned in pBluescript II SK(+) cut with EcoRV and treated with Antarctic Phosphatase. Clones containing the PCV2 ORF2 were sequenced using T7 and T3 primers as described above. The sequences were analyzed and compiled using Align, LAlign, ClustalW and Sixframe in the workbench (workbench.sdsc.edu) and Align2sequences, BlastN and BlastP at www.ncbi.nlm.nih.gov. Phylogenetic relationships among sequences were analyzed as described by Tripathi & Sowdhamini (2006). Briefly, phylogenetic trees were derived from multiple sequence alignments with PHYLIP version 3.67. Bootstrapping was performed 100 times using SEQBOOT. Pairwise distances between genomic sequences and protein sequences were determined with DNADIST and PROTDIST respectively. Neighbor-Joining (NJ) trees were calculated with NEIGHBOR and Maximum Likelihood (ML) trees with DNAML and PROML. Majority rule consensus trees were obtained with CONSENSE and visualized with DRAWGRAM. The ORF2 sequences (from ATG-stop: 702 nt for strains 1206 and VC2002-k39; 705 nt for strain VC2002-k2) from strains 1206 (SEQ ID Nos 5&6), VC2002-k39 (SEQ ID Nos 7&8) and VC2002-k2 (SEQ ID Nos 9&10) are provided in FIGS. 4 to 6 below.

### Results

### Mouse Immunisation

Prior to immunisation, 4 Balb/c mice were made immunotolerant to PK-15 cells by repeated injection of PCV negative PK-15 cells and cyclophosphamide. After this treatment, no or little reaction to PK-15 cells was observed on IPMA. All serum samples taken before immunisation were negative for anti-PCV2 antibodies as determined by IPMA. Two weeks after the first immunisation, all mice had anti-Stoon-1010 Ab titres between 2,560 and 40,960. One mouse with an IPMA Ab titre of 10,240 and without reaction to PK-15 cells was selected. It received a boost injection one week later and its spleen was used for the production of hybridomas.

### Production and Screening of Hybridomas

Forty-four hybridomas that produced mAbs against PCV2 infected PK-15 cells were frozen. Cloning by limiting dilution resulted in 16 stable PCV2-specific Ab-producing hybridomas with an IPMA titre of 1,000 or more to Stoon-1010.

### Determination of Monoclonal Antibody Class

A commercial identification kit was used to determine the isotypes of the mAbs. This is presented in Table 2. Six hybridomas produced IgG1 Abs and 8 hybridomas produced IgG2a Abs. MAb 21C12 had an IgG3 isotype. The isotype of mAb 48B5 could not be determined All mAbs, including mAb 48B5 had a light chain of the K-type.

### Indirect Immunofluorescence Staining of Recombinant PCV2 Virus-Like Particles

The reactivity of the mAbs to VLPs was tested by performing an indirect immunofluorescence staining on VLPs that were smeared onto glass slides. All 16 mAbs reacted with the VLPs indicating that the mAbs were directed against the PCV2 capsid protein. No staining was observed with irrelevant mAbs.

### Western Blot Analysis

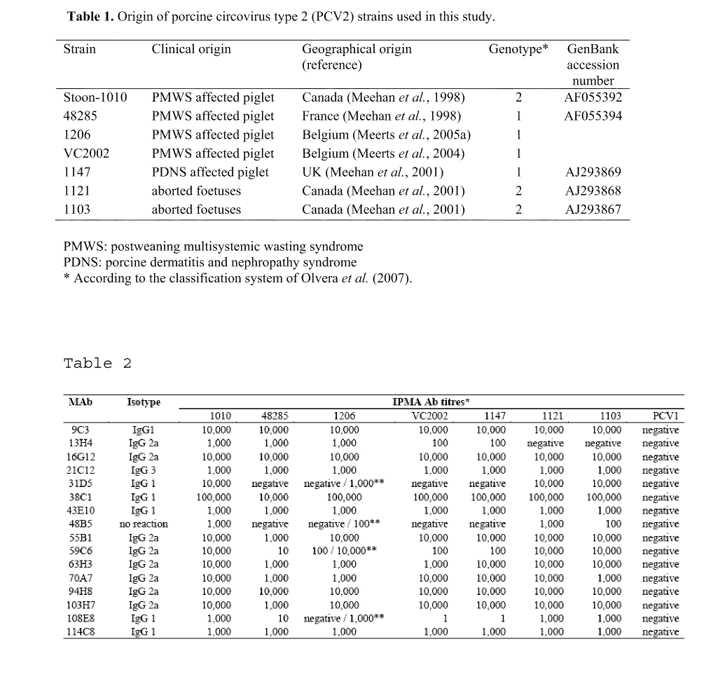

The reactivity of the mAbs to Stoon-1010 inoculated PK-15 cells was determined in a western blot assay. MAbs 31D5, 38C1 and 108E8 gave a strong and specific reaction with a protein of approximately 28 kDa. This is demonstrated in FIG. 1. For mAb 21C12 a faint but specific band was observed at 28 kDa. None of the other mAbs showed reactivity in the western blot assay.

### Reactivity of Monoclonal Antibodies to Different PCV2 Strains

An IPMA was used to examine the reactivity of hybridoma supernatants to 7 different PCV2 strains (Table 2). Eleven out of 16 hybridomas stained all 7 strains with a maximum 10-fold variation in titres in between the strains (9C3, 16G12, 21C12, 38C1, 43E10, 55B1, 63H3, 70A7, 94H8, 103H7 and 114C8). MAbs 31D5, 48B5, 325 59C6 and 108E8 did not react with the genotype 1 strains 48285, VC2002 and 1147 or they had IPMA Ab titres to these strains that were at least 100 times lower than for the genotype 2 strains Stoon-1010, 1121 and 1103. These 4 mAbs stained 2 different populations of infected cells in strain 1206. IPMA Ab titres for the first population (approximately 99% of the infected cells) were comparable to those of the other genotype 1 strains. IPMA Ab titres for the second population (approximately 1% of the infected cells) were comparable to those of the genotype 2 strains. MAb 13H4 stained all 4 PMWS-associated (Stoon-1010, 48285, 1206 and VC2002) and the single PDNS-associated strain (1147) but did not react with the 2 reproductive failure associated strains (1121 and 1103). None of the 16 mAbs reacted with PCV1 or PK-15 cells.

### Sensitive Neutralization Assays

A sensitive neutralization assay was used to determine the neutralizing activity of hybridoma supernatants. Table 3 shows the neutralization % with the standard deviations of the different mAbs. The neutralizing activities of mAbs 13D12 and 1C11 were 7±19% and −1±14% respectively. Because the mean neutralizing activity of mAb 13D12+ its standard deviation was 7+19=26%, a mAb was arbitrarily considered as neutralizing when its mean neutralizing activity was higher than 30%. The 11 mAbs (9C3, 16G12, 21C12, 38C1, 43E10, 55B1, 63H3, 70A7, 94H8, 103H7 and 114C8) that reacted equally with all 7 PCV2 strains in the IPMA demonstrated neutralization to Stoon-1010 (up to 95%), 48285 (up to 94%), 1206 (up to 57%) and 1103 (up to 61%). The 4 mAbs (31D5, 48B5, 59C6 and 108E8) that had a higher affinity for genotype 2 strains than for genotype 1 strains in the IPMA demonstrated neutralization to the genotype 2 strains Stoon-1010 (up to 98%) and 1103 (up to 67%). For these 4 mAbs, neutralization of the genotype 1 strains 48285 and 1206 was absent or very low (up to 35%). MAb 13H4 did not neutralize any of the 7 tested strains. Only one mAb (21C12) demonstrated some neutralization (32%) to strain VC2002 and only two mAbs (9C3 and 38C1) demonstrated some neutralization (34% and 30% respectively) to strain 1147. None of the 16 mAbs neutralized strain 1121.

Sequencing of ORF2 from Strains 1206 and VC2002

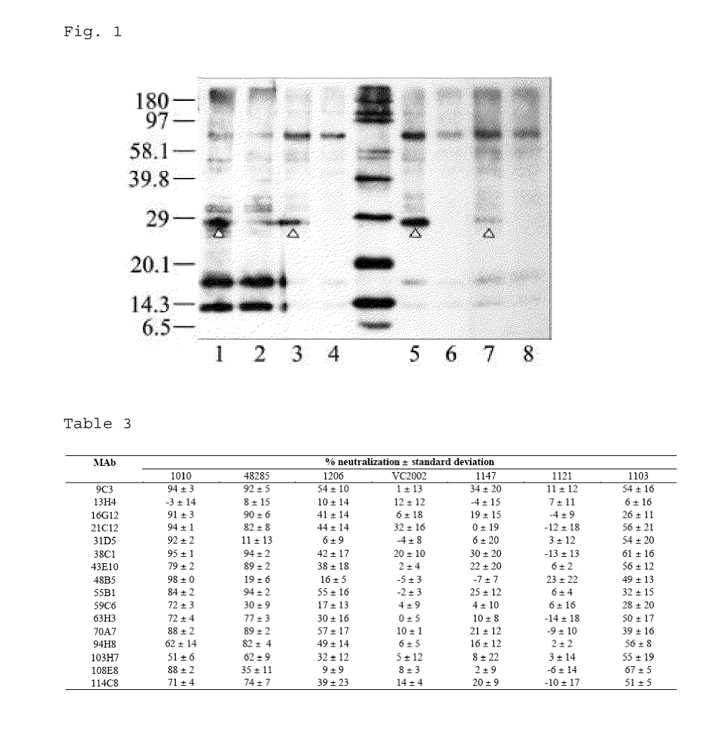

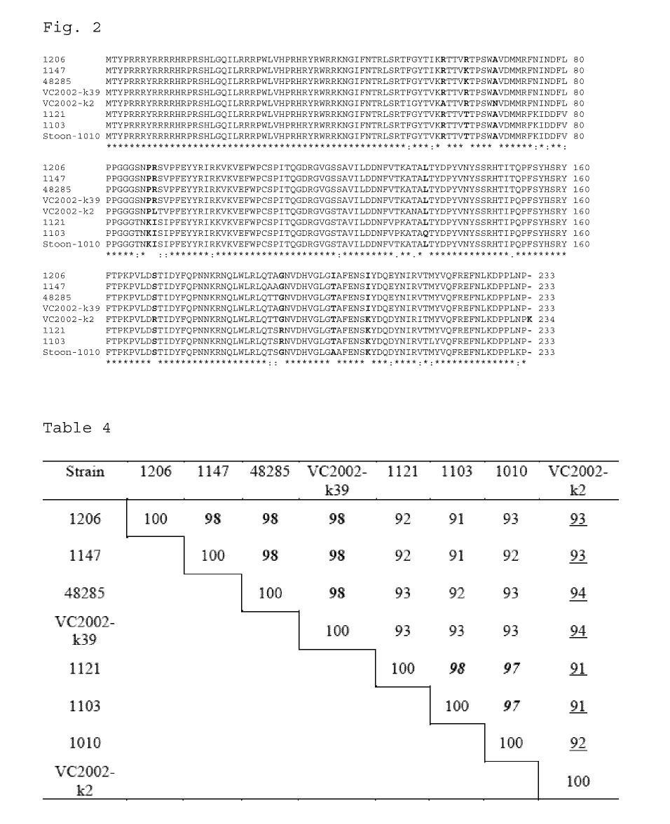

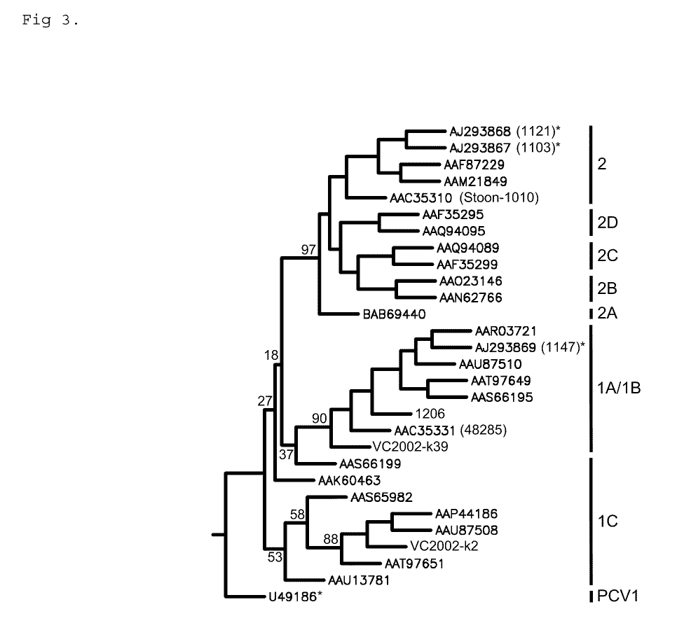

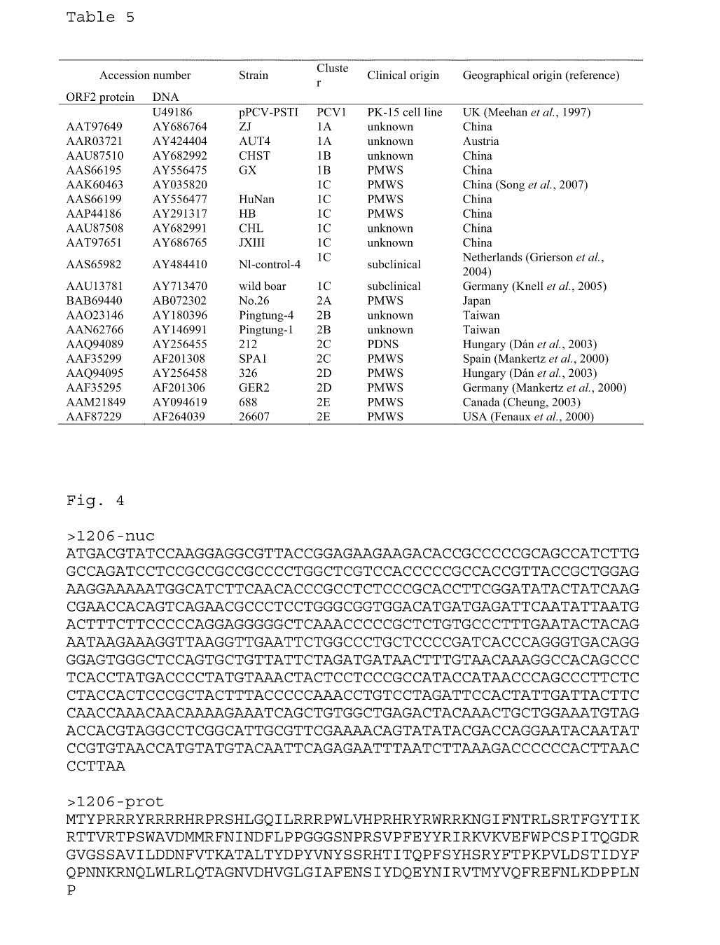

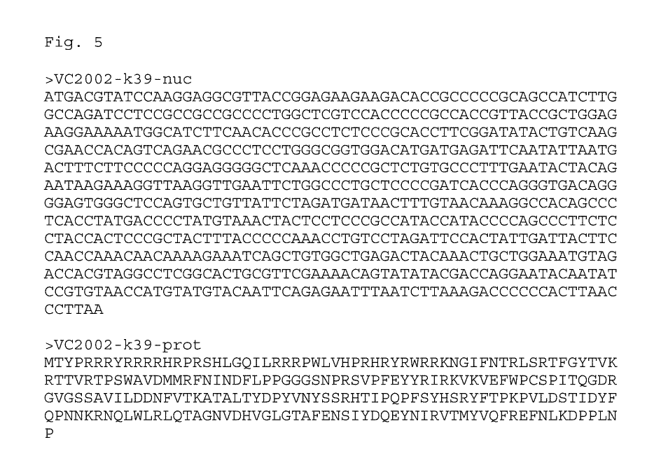

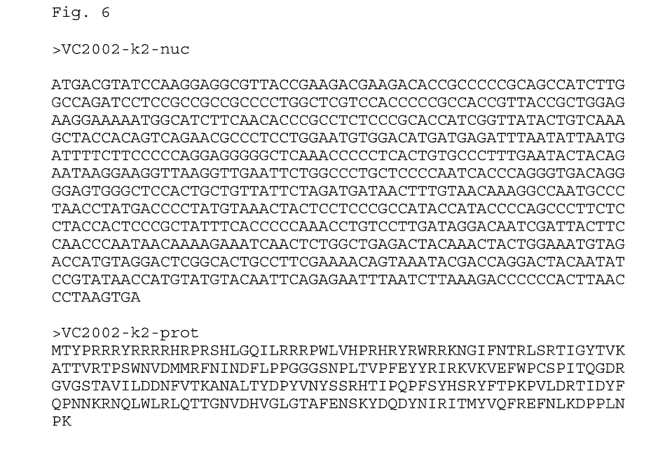

The ORF2 of the Belgian PMWS-associated PCV2 strains 1206 and VC2002 was amplified by PCR and sequenced. Strain 1206 contained an ORF2 of 702 by (starting from ATG including stop codon) encoding a 233 amino acid (aa) protein. Sequencing of the VC2002 ORF2 PCR product resulted in a sequence containing ambiguities at different positions. Therefore the VC2002 PCR fragment was cloned in pBluescript II SK(+) and 12 clones were sequenced. Clone VC2002-k39 contained an ORF of 702 by (starting from ATG including stop codon) encoding a protein of 233 aa. Ten other VC2002 clones were almost 100% identical at nucleotide (nt) level with k39 with 1-3 nt differences. Clone VC2002-k2 contained an ORF of 705 by (starting from ATG including stop codon) encoding a protein of 234 aa. Clone VC2002-k2 showed 94% identity with k39 at nt and aa level and 96-99% aa identity with strains from China (e.g. AAP44186, AAU87508, AAT97651), The Netherlands (AAS65982, Grierson et al., 2004) and a strain isolated from wild boars in Germany (AAU13781, Knell et al., 2005). Capsid protein similarity amongst the 7 different strains used in this study was determined using pairwise alignments and ClustalW (FIG. 2). The ORF2 aa identity of the strains that were used in this study is demonstrated in Table 4. FIG. 3 shows a phylogenetic tree of the ORF2 protein based on the NJ method with the percentages of confidence along the branches. This figure was constructed with ORF2 protein sequences from this study and sequences chosen from the different clusters from Olvera et al. (2007). The latter sequences are shown in Table 5. Genotype 1 strains 48285, 1206, VC2002-k39 and 1147 were assigned to cluster 1A/1B, VC2002-k2 to cluster 1C and genotype 2 strains Stoon-1010, 1121 and 1103 to cluster 2E. The same strain classification was obtained with the ML method and with ORF2 DNA sequences.

### Discussion

This is the first study that demonstrates antigenic diversity among PCV2 strains. This was established by the production and characterization of mAbs directed to the PCV2 capsid protein. The cross-reactivity of the mAbs to 7 different PCV2 strains with a different genotype and originating from various clinical conditions was determined Eleven mAbs (9C3, 16G12, 21C12, 38C1, 43E10, 55B1, 63H3, 70A7, 94H8, 103H7 and 114C8) reacted equally with the 7 PCV2 strains that were enclosed in the IPMA. Four other mAbs (31D5, 48B5, 59C6 and 108E8) were able to differentiate the genotype 1 strains 48285, 1206, VC2002 and 1147 from the genotype 2 strains Stoon-1010, 1121 and 1103 by IPMA, since they did not react with genotype 1 strains or had a reduced affinity compared to genotype 2 strains. The IPMA results of the latter 4 mAbs were also reflected in the neutralization assays. Until now, mAbs did not allow to differentiate PCV2 strains (Allan et al., 1999; McNeilly et al., 2001). MAbs 31D5, 48B5, 59C6 and 108E8 did also not react with or had a reduced affinity for tissue sections originating from the Belgian PMWS-affected pig from which the VC2002 strain was isolated. This was demonstrated by immunofluorescence staining and suggests that the results obtained by IPMA for mAbs 31D5, 48B5, 59C6 and 108E8 were not a consequence of PCV2 cell culture adaptation (data not shown). Using the IPMA, mAbs 31D5, 48B5, 59C6 and 108E8 stained 2 different populations of infected cells in strain 1206. This suggests that the 1206 strain consists of 2 viral subpopulations, where 99% of the virus behaves as a genotype 1 strain and 1% of the virus behaves as a genotype 2 strain. No signs of the existence of subpopulations were detected by sequencing strain 1206. This may be explained by the fact that the putative genotype 2 subpopulation was present at a very low level (1%). Sequencing of the VC2002 strain did reveal the existence of 2 PCV2 subpopulations in the virus stock. After cloning, 2 distinct sequences were derived from strain VC2002. Phylogenetic analysis assigned clone VC2002-k39 to cluster 1A/1B and demonstrated clustering of clone VC2002-k2 with strains from China, The Netherlands (Grierson et al., 2004) and a strain isolated from wild German boars (Knell et al., 2005), which documents the putative epidemiological link between PCV2 infections in domestic and wild pigs (Cságola et al., 2006). The identification of 2 different PCV2 sequences in one animal has been reported previously (de Boisséson et al., 2004; Opriessnig et al., 2006; Cheung et al., 2007), but the role of multiple PCV2 infections in the pathogenesis of PCV2-associated diseases is not clear.

Using protein sequences (NJ and ML), we were not able to differentiate between clusters 1A and 1B and not all sequences that were previously classified as 1C (Olvera et al., 2007) were found in the 1C cluster. Using the corresponding DNA sequences (NJ and ML), the same topology was obtained as Olvera et al. (2007), with the only difference that clusters 1A and 1B could not be differentiated in the present study (data not shown). We assume that these differences were a consequence of the reduced number of sequences that was used.

Putative amino acid substitutions that discriminate the genotype 1 strains 48285, 1206, VC2002-k39 and 1147 from the genotype 2 strains Stoon-1010, 1121 and 1103 are located at positions 63, 88, 89 and 206. At position 63, a threonine (T) was substituted for a lysine (K) or an arginine (R). At position 88 a lysine (K) was replaced by a proline (P) and at position 89 an isoleucine (I) was replaced by an arginine (R). These 3 substitutions all involve the basic aa K and R. Due to the differences in size, charge and hydrophobicity between K/R and T, P and I, this may have major consequences on the secondary and tertiary structure of the PCV2 capsid protein. The same comments can be made for position 206, where a lysine (K) was replaced by an isoleucine (I). Linear antigenic determinants of the PCV2 ORF2 protein, as determined by PEPSCAN, are located at positions 65-87, 113-139, 169-183 and 193-207 (Mahé et al., 2000). The positions 63, 88 and 89, where non-conserved mutations were found in the present study, are located at the outer borders of linear epitope 65-87, whereas position 206, where another non-conserved mutation was found, is located at the inner border of linear epitope 193-207. Therefore, we speculate that the aa substitutions that involve basic aa at positions 63, 88, 89 and 206 might be responsible for the fact that mAbs 31D5, 48B5, 59C6 and 108E8 did not react with the genotype 1 strains or that they had a reduced affinity for these strains in the IPMA and neutralization assay. This study also demonstrated that mAb 13H4 did not react specifically with the reproductive failure-associated strains 1121 and 1103 in the IPMA. Strains 1121 and 1103 have a proline at position 131 instead of a threonine (T131P), and an arginine instead of a glycine at position 191 (G191R). Proline is known to be a helix-breaker and glycine has a great conformational flexibility. Apart from the changes in the primary structure of the protein, T131P and G191R may have important consequences on the secondary and tertiary structure of a protein. Position 131 is located within and position 191 is located at the outer border of an antigenic domain (Mahé et al., 2000). Therefore, the substitutions at positions 131 and 191 might be involved in the absence of reaction of mAb 13H4 with strains 1121 and 1103. Previously, it was demonstrated by Meerts et al. (2005a) that the production of infectious virus in PK-15 cells is more efficient for Stoon-1010 than for strain 1121. Fenaux et al. (2004) demonstrated that PCV2 that was passaged 120 times in PK-15 cells (VP120) replicates more efficiently in PK-15 cells than wild type virus (VP1). Differences between VP1 and VP120 were a mutation from proline to alanine at position 110 (P110A) and a mutation from arginine to serine at position 191 (R191S). This may suggest that basic aa residues at position 191 influence not only mAb reactivity, but also the production of infectious virus.

Recently, it was demonstrated that PMWS-affected animals are not able to produce neutralizing Abs, whereas their ability to produce non-neutralizing Abs remains unaffected (Meerts et al., 2005b; Meerts et al., 2006; Fort et al., 2007). In these studies, it was suggested that PMWS-affected animals mount an immune response to non-neutralizing epitopes but not to neutralizing epitopes. In the present study, none of the tested mAbs was able to neutralize all 7 PCV2 strains, suggesting that a universal PCV2 neutralizing epitope does not exist. Neutralization was observed to Stoon-1010, 48285, 1206 and 1103, but not to VC2002, 1147 and 1121. No discriminative aa motifs that could explain these results were detected. The mAbs that neutralize Stoon-1010, 48285, 1206 and 1103 did not differentiate these strains from VC2002, 1147 and 1121 in the IPMA, indicating that 2 different groups of PCV2 strains have different neutralizing epitopes, and suggesting that these 2 different groups of PCV2 strains use different entry pathways in PK-15 cells. Recently, the glycosaminoglycans (GAGs) heparan sulfate and chondroitin sulfate B have both been described as attachment receptors for PCV2 (Misinzo et al., 2006). Protein binding to these 2 attachment receptors is restricted to the basic aa lysine (K) and arginine (R) (Esko, 1999), suggesting a crucial role of basic aa residues in the entry of PCV2 into the host cell. Positive aa charges of K and R interact three-dimensionally with negatively charged GAG sulfates and carboxylates (Esko, 1999), which indicates that three-dimensional conformation plays a crucial role in interactions between the PCV2 capsid protein and its receptors.

Until now, it was assumed that no distinct antigenic variation existed among PCV2 isolates. In this study, we clearly demonstrate the existence of major antigenic differences between the capsid proteins of PCV2 strains with a different genotype and isolated from different clinical presentations.

### REFERENCES

Allan G M, McNeilly F, Kennedy S, Daft B, Clark E G, Ellis J A, Haines D M, Meehan B M, Adair B M (1998) Isolation of porcine circovirus-like viruses from pigs with a wasting disease in the USA and Europe. J Vet Diagn Invest 10: 3-10
Allan G M, McNeilly F, Meehan B M, Kennedy S, Mackie D P, Ellis J A, Clark E G, Espuna E, Saubi N, Riera P, Bøtner A, Charreyre C E (1999) Isolation and characterisation of circoviruses from pigs with wasting syndromes in Spain, Denmark and Northern Ireland. Vet Microbiol 66: 115-123
Allan G M, Ellis J A (2000) Porcine circoviruses: a review. J Vet Diagn Invest 12: 3-14
Cheung A K (2003) Transcriptional analysis of porcine circovirus type 2. Virology 305: 168-180
Cheung A K, Lager K M, Kohutyuk D I, Vincent A L, Henry S C, Baker R B, Rowland R R, Dunham A G (2007) Detection of two porcine circovirus type 2 genotypic groups in United States swine herds. Arch Virol 152: 1035-1044
Cságola A, Kecskeméti S, Kardos G, Kiss I, Tuboly T (2006) Genetic characterization of type 2 porcine circoviruses detected in Hungarian wild boars. Arch Virol 151: 495-507
Dán A, Molnár T, Biksi I, Glávits R, Shaheim M, Harrach B (2003) Characterisation of Hungarian porcine circovirus 2 genomes associated with PMWS and PDNS cases. Acta Vet Hung 51: 551-562
de Boisséson C, Beven V, Bigarre L, Thiery R, Rose N, Eveno E, Madec F, Jestin A (2004) Molecular characterization of Porcine circovirus type 2 isolates from post-weaning multisystemic wasting syndrome-affected and non-affected pigs. J Gen Virol 85: 293-304
Delputte P L, Vanderheijden N, Nauwynck H J, Pensaert M B (2002) Involvement of the matrix protein in attachment of porcine reproductive and respiratory syndrome virus to a heparinlike receptor on porcine alveolar macrophages. J Virol 76: 4312-4320
Esko J D (1999) Glycosaminoglycan-binding Proteins. In Essentials of Glycobiology, pp. 441-453. Edited by A. Varki, R. Cummings, J. Esko, H. Freeze, G. Hart & J. Marth. New York: Cold Spring Harbor Laboratory Press.
Fenaux M, Halbur P G, Gill M, Toth T E, Meng X J (2000) Genetic characterization of type 2 porcine circovirus (PCV-2) from pigs with postweaning multisystemic wasting syndrome in different geographic regions of North America and development of a differential PCR-restriction fragment length polymorphism assay to detect and differentiate between infections with PCV-1 and PCV-2. J Clin Microbiol 38: 2494-2503
Fenaux M, Opriessnig T, Halbur P G, Elvinger F, Meng X J (2004) Two amino acid mutations in the capsid protein of type 2 porcine circovirus (PCV2) enhanced PCV2 replication in vitro and attenuated the virus in vivo. J Virol 78: 13440-13446
Fort M, Olvera A, Sibila M, Segales J, Mateu E (2007) Detection of neutralizing antibodies in postweaning multisystemic wasting syndrome (PMWS)-affected and non-PMWS-affected pigs. Vet Microbiol 125: 244-255
Galfre G, Milstein C (1981) Preparation of monoclonal antibodies: strategies and procedures. Methods Enzymol 73: 1-46
Grierson S S, King D P, Wellenberg G J, Banks M (2004) Genome sequence analysis of 10 Dutch porcine circovirus type 2 (PCV-2) isolates from a PMWS case-control study. Res Vet Sci 77: 265-268
Hamel A L, Lin L L, Nayar G P, (1998) Nucleotide sequence of porcine circovirus associated with postweaning multisystemic wasting syndrome 580 in pigs. J Virol 72:5262-5267
Knell S, Willems H, Hertrampf B, Reiner G (2005) Comparative genetic characterization of Porcine Circovirus type 2 samples from German wild boar populations. Vet Microbiol 109: 169-177
Labarque G G, Nauwynck H J, Mesu A P, Pensaert M B (2000) Seroprevalence of porcine circovirus types 1 and 2 in the Belgian pig population. Vet Quart 22: 234-236
Liu J, Chen I, Du Q, Chua H, Kwang J (2006) The ORF3 protein of porcine circovirus type 2 is involved in viral pathogenesis in vivo. J Virol 80: 5065-5073
Mahé D, Blanchard P, Truong C, Arnauld C, Le Cann P, Cariolet R, Madec F, Albina E, Jestin A (2000) Differential recognition of ORF2 protein from type 1 and type 2 porcine circoviruses and identification of immunorelevant 630 epitopes. J Gen Virol 81: 1815-1824
Mankertz A, Domingo M, Folch J M, Le Cann P, Jestin A, Segales J, Chmielewicz B, Plana-Duran J, Soike D (2000) Characterisation of PCV-2 isolates from Spain, Germany and France. Virus Res 66: 65-77
Matthew W D, Sandrock A W (1987) Cyclophosphamide treatment used to manipulate the immune response for the production of monoclonal antibodies J Immunol Methods 100: 73-82
McNeilly F, McNair I, Mackie D P, Meehan B M, Kennedy S, Moffett D, Ellis J, Krakowka S, Allan G M (2001) Production, characterization and applications of monoclonal antibodies to porcine circovirus 2. Arch Virol 146: 909-922
Meehan B M, Creelan J L, McNulty M S, Todd D (1997) Sequence of porcine circovirus DNA: affinities with plant circoviruses. J Gen Virol 78: 221-227
Meehan B M, McNeilly F, Todd D, Kennedy S, Jewhurst V A, Ellis J A, Hassard L E, Clark E G, Haines D M, Allan G M (1998) Characterization of novel circovirus DNAs associated with wasting syndromes in pigs. J Gen Virol 79: 2171-2179
Meehan B M, McNeilly F, McNair I, Walker I, Ellis J A, Krakowka S, Allan G M, (2001) Isolation and characterization of porcine circovirus 2 from cases of sow abortion and porcine dermatitis and nephropathy syndrome. Arch Virol 146: 835-842
Meerts P, Nauwynck H, Sanchez R, Mateusen B, Pensaert, M (2004) Prevalence of porcine circovirus 2 (PCV2)-related wasting on Belgian farms with or without a history of postweaning multisystemic wasting syndrome. Vlaams Diergen Tijds 73: 31-38
Meerts P, Misinzo G, McNeilly F, Nauwynck H J (2005a) Replication kinetics of different porcine circovirus 2 strains in PK-15 cells, foetal cardiomyocytes and macrophages. Arch Virol 150: 427-441
Meerts P, Van Gucht S, Cox E, Vandebosch A, Nauwynck H J (2005b) Correlation between type of adaptive immune response against porcine circovirus type 2 and level of virus replication. Viral Immunol 18: 333-341
Meerts P, Misinzo G, Lefebvre D, Nielsen J, Bøtner A, Kristensen C S, Nauwynck H J (2006) Correlation between the presence of neutralizing antibodies against porcine circovirus 2 (PCV2) and protection against replication of the virus and development of PCV2-associated disease. BMC Vet Res 2, 6.
Misinzo G, Meerts P, Bublot M, Mast J, Weingartl H M, Nauwynck H J (2005) Binding and entry characteristics of porcine circovirus 2 in cells of the porcine monocytic line 3D4/31. J Gen Virol 86: 2057-2068
Misinzo G, Delputte P L, Meerts P, Lefebvre D J, Nauwynck H J (2006) Porcine circovirus 2 uses heparan sulfate and chondroitin sulfate B glycosaminoglycans as receptors for its attachment to host cells. J Virol 80: 3487-3494
Nauwynck H J, Pensaert M B (1995) Effect of specific antibodies on the cellassociated spread of pseudorabies virus in monolayers of different cell types. Arch. Virol 140: 1137-1146
Nawagitgul P, Morozov I, Bolin S R, Harms P A, Sorden S D, Paul P S (2000) Open reading frame 2 of porcine circovirus type 2 encodes a major capsid protein. J Gen Virol 81: 2281-2287
Olvera A, Cortey M, Segales J (2007) Molecular evolution of porcine circovirus type 2 genomes: Phylogeny and clonality. Virology 357: 175-185
Opriessnig T, McKeown N E, Zhou E M, Meng X J, Halbur P G (2006) Genetic and experimental comparison of porcine circovirus type 2 (PCV2) isolates from cases with and without PCV2-associated lesions provides evidence for differences in virulence. J Gen Virol 87: 2923-2932
Pensaert M B, Sanchez R E, Ladekjer-Mikkelsen A S, Allan G M, Nauwynck H J (2004) Viremia and effect of fetal infection with porcine viruses with special reference to porcine circovirus 2 infection. Vet Microbiol 98: 175-183
Song Y, Jin M, Zhang S, Xu X, Xiao S, Cao S, Chen H (2007) Generation and immunogenicity of a recombinant pseudorabies virus expressing cap protein of porcine circovirus type 2. Vet Microbiol 119: 97-104
Tiels P, Verdonck F, Coddens A, Ameloot P, Goddeeris B, Cox E (2007) Monoclonal antibodies reveal a weak interaction between the F18 fimbrial adhesin FedF and the major subunit FedA. Vet Microbiol, 119: 115-120
Tischer I, Mields W, Wolff D, Vagt M, Griem W (1986) Studies on epidemiology and pathogenicity of porcine circovirus. Arch Virol 91: 271-276
Tripathi, L. P., Sowdhamini, R. (2006). Cross genome comparisons of serine proteases in Arabidopsis and rice. BMC Genomics 7, 200.
West, K. H., Bystrom, J. M., Wojnarowicz, C., Shantz, N., Jacobson, M., Allan, G. M., Haines, D. M., Clark, E. G., Krakowka, S., McNeilly, F., Konoby, C., Martin, K., Ellis, J. A. (1999). Myocarditis and abortion associated with intrauterine infection of sows with porcine circovirus 2. J Vet Diagn Invest 11, 530-532.

1. A method to assign PCV2 strains to serogroups said method comprising contacting a sample with an antibody that specifically or selectively binds to immunogenic regions in the capsid protein of the porcine circovirus comprising one or more polypeptides selected from the group consisting of
a. immunogenic variant capsid proteins of the PCV2 strains 1206 (SEQ ID NO: 6) and VC2002 (SEQ ID NO: 8 and NO:10);
b. an immunogenic fragment of said capsid proteins comprising at least one of the epitopes selected from the polypeptides; YTVKRTTVTTPSWAV, GGTNKISIPFEY, AFENSKYDQDY, DNFYTKATALTYD and RLQTSGNVDHV; and
c. variants thereof that have at least 70%, 80%, 85%, 90%, 95%, 96%, 97%, 98%, 99% identity to the polypeptides of b); and
detecting the binding of the antibody to any one of said polypeptides.

2. The method according to claim L wherein the serogroups differ in genotype or differ in pathogenicity.

3. The method according to claim 2, wherein the difference in PCV2 genotype is determined using an antibody that specifically or selectively binds to one or more of the polypeptide(s) selected from the group consisting of;
a. immunogenic variant capsid proteins of the PCV2 strains 1206 (SEQ ID NO:6) and VC2002 (SEQ ID NO:8 and SEQ ID NO: 10);
b. an immunogenic fragment of said capsid proteins comprising at least one of the epitopes selected from the polypeptides; YTVKRTTVTTPSWAV, GGTNKISIPFEY, and AFENSKYDQDY;
c. variants thereof that have at least 70%, 80%, 85%, 90%, 95%, 96%, 97%, 98%, 99% identity to the polypeptides of b); and
detecting the binding of the antibody to any one of said polypeptides.

4. The method according to claim 3, wherein the antibody is a monoclonal antibody.

5. The method according to claim 2, wherein the difference in pathogenicity is determined using an antibody that specifically or selectively binds to one or more polypeptides selected from the group consisting of;
a. immunogenic variant capsid proteins of the PCV2 strains 1206 (SEQ ID NO: 6) and VC2002 (SEQ ID NO: 8 and SEQ ID NO: 10);
b. an immunogenic fragment of said capsid proteins comprising at least one, in particular two of the epitopes selected from the polypeptides; DNFYTKATALTYD and RLQTSGNVDHV;
c. variants thereof that have at least 70%, 80%, 85%, 90%, 95%, 96%, 97%, 98%, 99% identity to the polypeptides of b); and
detecting the binding of the antibody to any one of said polypeptides;

6. The method according to claim 5, wherein the antibody is a monoclonal antibody.

7. The method according to claim 1, wherein the sample is selected from a biological fluid, tissue or a tissue extract, freshly harvested cells, or lysates of cells which have been incubated in cell culture.

8. An antibody for use in a method according to claim 1, wherein said antibody is specific for a polypeptide as claimed in claim 1.

9. The antibody according to claim 8, wherein said antibody is a monoclonal antibody.

10. A kit to identify antigenic differences between PCV2 strains said kit comprising one or more antibodies as defined in claim 1.

11. The kit according to claim 10, wherein said antibodies are selected from the group consisting of the monoclonal antibodies 13H4, 31D5, 48B5, 59C6 and 108E8.

12-17. (canceled)

18. A method for serotyping a PCV2 infection or a previous PCV2 infection said method comprising,
contacting an antigen comprising, immunogenic capsid PCV2 proteins, immunogenic fragments or polypeptides according to claim 1; with a fluid sample; and
determining the presence of an antigen-serum antibody complex.

19. The method according to claim 18, wherein the presence of an antigen-serum antibody complex is determined using the monoclonal antibodies as claimed in claim 9.

20. A method of isolating PCV2 serotypes comprising using the monoclonal antibody as claimed in claim 9 in isolating PCV2 serotypes.

21. (canceled)

22. The method according to claim 2, wherein differences in genotype include differences in geographic origin and differences in pathogenicity including differences in clinical representation.

23. The method according to claim 4, wherein the monoclonal antibody is selected from the group consisting of 31D5, 48B5, 59C6 and 108E8.

24. The method according to claim 6, wherein the monoclonal antibody is 13H4.

25. The antibody according to claim 9, wherein said monoclonal antibody is selected from the group consisting of 13H4, 31D5, 48B5, 59C6 and 108E8.


SEQUENCE LISTING (20 sequences)

SEQ ID NO: 1 - DNA, 22 bp, Porcine circovirus Type II
agcgcacttctttcgttttcag

SEQ ID NO: 2 - DNA, 22 bp, Porcine circovirus Type II
agcgcacttctttcgttttcag

SEQ ID NO: 3 - DNA, 19 bp, Porcine circovirus Type II
gggctgtggcctttgktac

SEQ ID NO: 4 - DNA, 20 bp, Porcine circovirus Type II
tgtrgaccacgtaggcctcg

SEQ ID NO: 5 - DNA, 702 bp, Porcine circovirus Type II
atgacgtatccaaggaggcgttaccggagaagaagacaccgcccccgcagccatcttggccagatcctccgccgccgcccctggctcgtccacccccgccaccgttaccgctggagaaggaaaaatggcatcttcaacacccgcctctcccgcaccttcggatatactatcaagcgaaccacagtcagaacgccctcctgggcggtggacatgatgagattcaatattaatgactttcttcccccaggagggggctcaaacccccgctctgtgccctttgaatactacagaataagaaaggttaaggttgaattctggccctgctccccgatcacccagggtgacaggggagtgggctccagtgctgttattctagatgataactttgtaacaaaggccacagccctcacctatgacccctatgtaaactactcctcccgccataccataacccagcccttctcctaccactcccgctactttacccccaaacctgtcctagattccactattgattacttccaaccaaacaacaaaagaaatcagctgtggctgagactacaaactgctggaaatgtagaccacgtaggcctcggcattgcgttcgaaaacagtatatacgaccaggaatacaatatccgtgtaaccatgtatgtacaattcagagaatttaatcttaaagaccccccacttaacccttaa

SEQ ID NO: 6 - protein, 233 aa, Porcine circovirus Type II
MTYPRRRYRRRRHRPRSHLGQILRRRPWLVHPRHRYRWRRKNGIFNTRLSRTFGYTIKRTTVRTPSWAVDMMRFNINDFLPPGGGSNPRSVPFEYYRIRKVKVEFWPCSPITQGDRGVGSSAVILDDNFVTKATALTYDPYVNYSSRHTITQPFSYHSRYFTPKPVLDSTIDYFQPNNKRNQLWLRLQTAGNVDHVGLGIAFENSIYDQEYNIRVTMYVQFREFNLKDPPLNP

SEQ ID NO: 7 - DNA, 702 bp, Porcine circovirus Type II
atgacgtatccaaggaggcgttaccggagaagaagacaccgcccccgcagccatcttggccagatcctccgccgccgcccctggctcgtccacccccgccaccgttaccgctggagaaggaaaaatggcatcttcaacacccgcctctcccgcaccttcggatatactgtcaagcgaaccacagtcagaacgccctcctgggcggtggacatgatgagattcaatattaatgactttcttcccccaggagggggctcaaacccccgctctgtgccctttgaatactacagaataagaaaggttaaggttgaattctggccctgctccccgatcacccagggtgacaggggagtgggctccagtgctgttattctagatgataactttgtaacaaaggccacagccctcacctatgacccctatgtaaactactcctcccgccataccataccccagcccttctcctaccactcccgctactttacccccaaacctgtcctagattccactattgattacttccaaccaaacaacaaaagaaatcagctgtggctgagactacaaactgctggaaatgtagaccacgtaggcctcggcactgcgttcgaaaacagtatatacgaccaggaatacaatatccgtgtaaccatgtatgtacaattcagagaatttaatcttaaagaccccccacttaacccttaa

SEQ ID NO: 8 - protein, 233 aa, Porcine circovirus Type II
MTYPRRRYRRRRHRPRSHLGQILRRRPWLVHPRHRYRWRRKNGIFNTRLSRTFGYTVKRTTVRTPSWAVDMMRFNINDFLPPGGGSNPRSVPFEYYRIRKVKVEFWPCSPITQGDRGVGSSAVILDDNFVTKATALTYDPYVNYSSRHTIPQPFSYHSRYFTPKPVLDSTIDYFQPNNKRNQLWLRLQTAGNVDHVGLGTAFENSIYDQEYNIRVTMYVQFREFNLKDPPLNP

SEQ ID NO: 9 - DNA, 702 bp, Porcine circovirus Type II
atgacgtatccaaggaggcgttaccgaagacgaagacaccgcccccgcagccatcttggccagatcctccgccgccgcccctggctcgtccacccccgccaccgttaccgctggagaaggaaaaatggcatcttcaacacccgcctctcccgcaccatcggttatactgtcaaagctaccacagtcagaacgccctcctggaatgtggacatgatgagatttaatattaatgattttcttcccccaggagggggctcaaaccccctcactgtgccctttgaatactacagaataaggaaggttaaggttgaattctggccctgctccccaatcacccagggtgacaggggagtgggctccactgctgttattctagatgataactttgtaacaaaggccaatgccctaacctatgacccctatgtaaactactcctcccgccataccataccccagcccttctcctaccactcccgctatttcacccccaaacctgtccttgataggacaatcgattacttccaacccaataacaaaagaaatcaactctggctgagactacaaactactggaaatgtagaccatgtaggactcggcactgccttcgaaaacagtaaatacgaccaggactacaatatccgtataaccatgtatgtacaattcagagaatttaatcttaaagaccccccacttaacccttaa

SEQ ID NO: 10 - protein, 233 aa, Porcine circovirus Type II
MTYPRRRYRRRRHRPRSHLGQILRRRPWLVHPRHRYRWRRKNGIFNTRLSRTIGYTVKATTVRTPSWNVDMMRFNINDFLPPGGGSNPLTVPFEYYRIRKVKVEFWPCSPITQGDRGVGSTAVILDDNFVTKANALTYDPYVNYSSRHTIPQPFSYHSRYFTPKPVLDRTIDYFQPNNKRNQLWLRLQTTGNVDHVGLGTAFENSKYDQDYNIRITMYVQFREFNLKDPPLNP

SEQ ID NO: 11 - protein, 15 aa, Porcine circovirus Type II
YTVKRTTVTTPSWAV

SEQ ID NO: 12 - protein, 13 aa, Porcine circovirus Type II
GGTNKISIPFEYY

SEQ ID NO: 13 - protein, 11 aa, Porcine circovirus Type II
AFENSKYDQDY

SEQ ID NO: 14 - protein, 13 aa, Porcine circovirus Type II
DNFYTKATALTYD

SEQ ID NO: 15 - protein, 11 aa, Porcine circovirus Type II
RLQTSGNVDHV

SEQ ID NO: 16 - DNA, 45 bp, Porcine circovirus Type II
tatactgtcaagcgtaccacagtcacaacgccctcctgggcggtg

SEQ ID NO: 17 - DNA, 36 bp, Porcine circovirus Type II
ggggggaccaacaaaatctctataccctttgaatac

SEQ ID NO: 18 - DNA, 33 bp, Porcine circovirus Type II
gcgttcgaaaacagtaaatacgaccaggactac

SEQ ID NO: 19 - DNA, 39 bp, Porcine circovirus Type II
gataactttgtaacaaaggccacagccctaacctatgac

SEQ ID NO: 20 - DNA, 33 bp, Porcine circovirus Type II
agactacaaacctctggaaatgtggaccacgta